(19) United States
(12) Patent Application Publication
(10) Pub. No.: US 2024/0000385 A1
(43) Pub. Date: Jan. 4, 2024

(54) Smart bracelet device containing glucose and/or glucagon

(71) Applicant: Cristina CUCCHIARELLI, ROMA (IT)

(72) Inventor: Cristina CUCCHIARELLI, ROMA (IT)

(21) Appl. No.: 18/252,604

(22) PCT Filed: Nov. 15, 2021
(86) PCT No.: PCT/IB2021/060542

§ 371 Date: May 11, 2023

(30) Foreign Application Priority Data
Nov. 16, 2020 (IT) 102020000027375

(51) Int. Cl. A61B 5/00; A61B 5/145; A61M 5/142; A61M 5/172

(52) U.S. Cl. CPC A61B 5/681; A61B 5/14532; A61B 5/4839; A61B 5/0022; A61M 5/14244; A61M 5/1723; A61B 5/7435; A61B 5/746

(57) ABSTRACT

“Smart bracelet device containing glucose and/or glucagon” Ergonomic, wearable smart device, containing predetermined quantities of glucose and/or glucagon adapted to restore the concentration of glucose in the blood comprising: —a body of the device (1) with tubular shape containing predetermined quantities of glucose and/or glucagon; —a reload valve (4) housing at least a lip (6.1) of a reload cartridge (6), adapted to restore the reserves of glucose and/or glucagon; —a straw (3), a nasal spray (11) or both adapted for the consumption of a predetermined quantity of glucose and/or glucagon; —a smart valve (2) adapted to receive information regarding the exact quantity to be administered; —a magnetic closure (5); —a reload cartridge (6) comprising a tank (6.2) and a lip (6.1); —a display (7); —a glycemia detector (8) adapted to check the quantity of glucose in the blood, adapted to interact with said display (7) with latest-generation cellular device, CGM, insulin pumps.

### FIELD OF THE ART

The present invention operates in the field of integrated devices and systems for protecting people. More in detail, within this industrial field, the finding described and claimed hereinbelow specifically regards a wearable device for monitoring and protection from hypoglycemia events.

### PRIOR ART

At the state of the art, there are a variety of devices which allow monitoring the concentration of glucose in the blood.

It is in fact of fundamental importance, in subjects affected by pathologies such as diabetes, to constantly monitor the glucose levels thereof and supplement them in a quick/sudden manner if necessary, in order to prevent the occurrence of a situation of hypoglycemia.

According to data published in 2017 by the World Diabetes Federation, worldwide there are over 415 million people who live with diabetes and this number is destined to increase to 642 million in 2040. The consequences of only one episode of severe hypoglycemia can have considerable consequences on cerebral functionality and constant hypoglycemic crises are associated with an increased risk of cardiovascular events and mortality. According to an English study of 1979, hypoglycemia would be responsible for 4% of deaths of people under 50 with diabetes; a recent Norwegian study instead estimates on the order of 8% the predominance of deaths directly linked to hypoglycemia in a type 1 diabetic population under 56 years old. The studies conducted worldwide on subjects with type 1 and 2 diabetes have detected a risk of cardiovascular events and mortality increased by 1.5 to 6 times in subjects with hypoglycemic crises. This association is present both in subjects under therapy with insulin and in those under treatment with sulfonylurea. Particularly at risk are elderly patients (>75 years) with diabetes, and fragile subjects with comorbidity and chronic pathologies in terminal phase. In this category, the benefits of a very strict metabolic control have not been demonstrated; on the contrary, this could expose them to increased risk, especially tied to hypoglycemia. This information is very well-known, yet notwithstanding this it is quite common that these patients are hyper-treated and this increases the risk of hypoglycemia (already per se associated with age), of a series of morbidities, of a reduction of the cognitive performances and of renal functionality.

In addition, severe hypoglycemic crises are quite frequent in pregnant diabetic women treated with insulin, especially in the first trimester and in the under 18 group the most probable effect of hypoglycemia on the cardiovascular system is sudden death during sleep (dead-in-bed-syndrome).

The evidence that hypoglycemia can determine heart dysfunction and sudden death (e.g. from arrythmias) is increasingly convincing. Hypoglycemia activates the sympathetic nervous system and determines the release of catecholamines which in turn increase the heart rate, the contractility and the cardiac output; the plasmatic potassium is quickly reduced, determining electrophysiological alterations and ECG alterations. Other alterations can also occur: of the coagulation, endothelial dysfunction and release of inflammatory cytokines; all alterations which can lead to intravascular coagulation and thrombosis. The hemorheological and inflammatory alterations induced by hypoglycemia can last for days in subjects with type 2 diabetes and the functional alterations, which persist even after the restoration of a normal glycemia, can determine an intravascular environment favorable for a thrombosis event. In subjects with type 2 diabetes, therefore, these alterations associated with hypoglycemia could be responsible for acute cardiovascular events, such as ischemia and heart attack, heart failure, heart arrythmias.

Hypoglycemia also leads to neuronal damage. The regions hit by a hypoglycemic crises are the cortex, the neostriatum and the hippocampus which can reach neuronal death. Hypoglycemia is an important risk factor for cognitive deterioration. Slight moderate hypoglycemia (70-40) produces a neuron shock; severe hypoglycemia can determine neuronal death. The presence of diabetes worsens the neuronal damage and the cognitive deficit induced by hypoglycemia.

This condition can also have considerable psychological effects, both on the patient and on his/her family members. In addition to negatively affecting the quality of life, the patient (or his/her family members) live in fear that this could repeat itself. This is the case for parents of children with type 1 diabetes (for which the episodes of nocturnal hypoglycemia of their children represent violent emotional stress), as with subjects with reduced awareness of the hypoglycemia episodes, which can lead to convulsions and coma without premonitory symptoms or with subjects who have experienced a hypoglycemia episode in public and are very much embarrassed by the event. Several patients react by maintaining the glycemia levels higher, in order to avoid further hypoglycemic crises, but in such a manner exposing themselves to poor metabolic control and without informing the doctor of this poor compliance with the therapies.

For those affected with diabetes who wish to undertake a physical activity, hypoglycemia can represent a serious risk: when the level of glucose in the blood falls below 40-50 mg/dL, in fact, one can even reach so-called hypoglycemic coma, which is generally manifested after an excessive physical force, following dietetic errors (late consumption or non-consumption of food), after insulin or oral glycemia-lowering drug administration errors, following situations that prolong the glycemia-lowering action of the insulin or of oral antidiabetic substances (reduced renal functionality or association with other drugs). The symptoms that precede the hypoglycemic coma are: dizziness, fatigue, sensation of hunger, sweating, tachycardia, tremors, agitation, headaches, lip formication, speech difficulty. Hypoglycemia can also appear even several hours after exercise.

In order to limit the onset of hypoglycemia events, presently there are many different monitoring systems such as, for example, the device “CGM Dexcom G5 Mobile” which is a continuous glycemia monitoring device recommended for detecting glycemic trends and patterns in adults and in children and young adults of pediatric age (2-17 years old) with Type 1 and Type 2 diabetes subjected to insulin therapy.

The CGM is composed of an electrochemical sensor positioned below the skin of the abdomen which uninterruptedly measures up to 288 levels of glucose each day (one every 5 minutes). It is provided with support base for housing the Dexcom G5 Mobile transmitted and the suitable applicator which facilitates the insertion thereof and renders it painless. The sensor G5 Mobile is a sterile disposable device with 7 day duration.

Another example is the patent WO2019099626A which describes a method for estimating a level of glucose concentration in the blood. The method includes the obtainment of a first and a second measurement of the glucose, the addition of the first and of the second glucose measurement to a set of data for the measurement of the glucose and the calculation of an estimated level of HbA1c. In addition, a method is provided for calculating an estimated HbA1c level interval. The method comprises at least a calculation of an estimated HbA1c level and a standard deviation of the estimated HbA1c level by using a set of data for measuring the glucose and combining the estimated HbA1c level with the standard deviation of the estimated HbA1c level in order to acquire the HbA1c interval. In other words, the device for monitoring the glucose can monitor the glycemic variability of an individual.

Another example is the patent CA3077994A1 which describes a kit that provides methods and devices for measuring and managing the analyses in order to inform a diabetic patient regarding the glycemic conditions. The method provides for the execution of a measurement of the glucose before a meal with the analysis measurement and management device, in which the measurement of the glucose before the meal is marked in the memory of the analysis measurement and management device as the pre-meal glucose value. A measurement of the glucose is executed after a meal with the analysis measurement and management device, where the measurement of the glucose after the meal is marked in the memory of the device as post-meal value. The method also provides for determining if the difference between the post-meal glucose value and the pre-meal glucose value is lower than about 50 mg/dL and for warning the user each time the difference is greater than about 50 mg/dL. In addition, it reminds the user to repeat the test at a later time period.

None of the devices present today on the market allows the immediate access to reserves of glucose so as to not incur problems deriving from hypoglycemia crises. By not supplementing glucose within a proper time period, in fact, the risk of incurring irreversible consequences would remain unchanged. Therefore, due to the device described in detail hereinbelow, it will be possible to have an effective and immediate restoration of the glucose in any daily life situation so as to eliminate the risks tied to hypoglycemia, as well as a considerable psychological relief of the subjected affected with such deficiency.

### DESCRIPTION OF THE INVENTION

According to the present invention, the ergonomic, wearable smart device is attained, containing glucose and/or glucagon adapted to restore the concentration of glucose in the blood. Due to the capacity of this device to store predefined quantities of glucose and/or glucagon, due to its tubular shape or any other shape that renders it wearable and due to an advantageous magnetic closure, there is the possibility to quickly/suddenly respond to cases of hypoglycemia. Advantageously, in addition, situated on the surface of the device itself is a valve for reloading glucose, which occurs due to the advantageous use of a reload cartridge, in turn divided into: tank and lip (unit which will penetrate the reload valve, allowing the reloading itself).

Advantageously in order to facilitate the consumption of the glucose, there is a reversibly liftable straw, which at its interior houses a smart valve, which, advantageously being able to communicate via Bluetooth with latest-generation cellular device, CGM and insulin pumps, allows metering the liquid contained in the device and simultaneously prevents the leakage thereof. If the device housed predefined quantities of glucagon, however, it will be possible to consume it by means of a nasal spray integrated with the device. If instead the device was designed to house both glucose and glucagon, the device would be equipped both with straw and spray so as to make possible the consumption of both substances by the user.

In addition, the device is provided with a display with movement and/or touch interaction technology which provides a series of information benefiting the user, and can also interact with the aforesaid smart valve and with a glycemia detector, it too situated on the device, which can advantageously measure the levels of glucose in the blood and in turn communicate with the display.

In a further version, the device is capable of communicating with multiple smart-phones, it allows remote interactions and can signal important information to the user by means of sounds and vibrations as well as geolocate the user himself/herself and send SOS messages in case of need.

Furthermore and advantageously, an injection device is integratable, constituted by a retractable needle adapted to act as syringe, capable of being activated by the display or by multiple electronic devices, also remotely, due to the use of an application that was suitably designed for managing and controlling the entire device.

In a further advantageous version, the device can be composed of materials which render it impermeable, fireproof and underwater as well as having dark color so as to prevent the deterioration of the liquid at its interior and more generally allow the maintenance of a suitable temperature in order to prevent the crystallization or oxidation of the liquid itself.

Possibly, the device is made with recycled, recyclable and reusable materials designed such that they can fall within a circular economy.

### DESCRIPTION OF THE FIGURES

The invention will be described hereinbelow in a preferred embodiment as a non-limiting example, with the aid of the enclosed figures, in which:

FIG. 1 shows several of the components of the present smart bracelet device: the body of the device 1 comprises a smart valve 2 inserted at the base of a straw 3 as well as a reload valve 4, a nasal spray 11, a closure 5 composed of a right end 1.1 and a left end 1.2, a reload cartridge 6 composed of a lip 6.1 and a tank 6.2; a display 7 and a glycemia detector 8 which are adjacent to each other.

FIG. 2 schematically shows a bracelet device 1 according to the present invention in which in proximity to the display 7, the retractable needle 7.1. is placed, and the communication network between the display 7 and the connected portable devices is also shown.

### DETAILED DESCRIPTION OF THE INVENTION

The present invention will now be illustrated merely as a non-limiting or non-constraining example, with reference to the figures which illustrate possible embodiments relative to the present inventive concept.

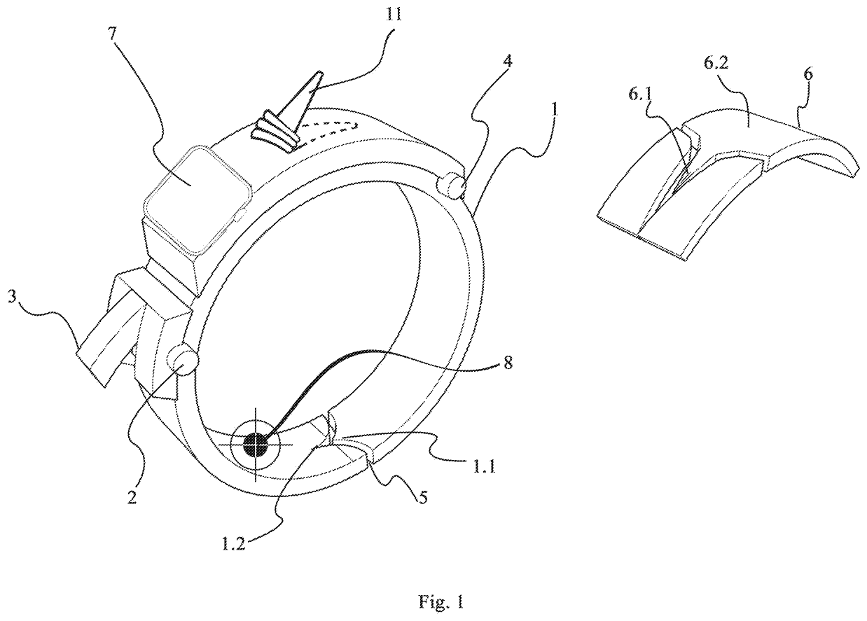

FIG. 1 shows the ergonomic, wearable smart device, containing glucose and/or glucagon in a preferred version: it is designed for restoring the concentration of glucose in the blood. The aforesaid wearable device is programmed for containing predetermined quantities of glucose and/or glucagon to be administered in case of episodes of severe hypoglycemia or hypoglycemia and comprises at least a body of the device 1 with tubular shape or with any other shape, adapted to contain predetermined quantities of glucose and/or glucagon. The aforesaid body of the device 1 is designed for being worn and fixed by means of a closure 5: this is designed for joining the two ends of the body of the device 1 which is designed for being reloaded by means of at least a reload valve 4 which allows the reloading of glucose and/or glucagon and simultaneously prevents the leakage thereof.

The aforesaid reload valve 4 is designed for allowing the insertion of at least a lip 6.1 of a reload cartridge 6; the valve 4, in addition, is designed for allowing the restoration of the reserves of glucose and/or glucagon, allowing the containment and the preservation of the same within the body device 1.

At least a straw 3, reversibly liftable, is placed above the aforesaid body of the device 1 and allows the consumption of a predetermined quantity of glucose.

In an embodiment variant, the device 1 of the present invention comprises glucagon and a retractable nasal spray 11 adapted to allow the nasal spraying of the content of the device 1, in case of need, by the user who wears the device on the wrist.

In a further variant, the aforesaid device 1 simultaneously comprises at its interior predefined quantities of glucose and glucagon, as well as at least a straw 3 and at least a nasal spray 11 which allow the consumption of both substances by the user.

In addition, at least a smart valve 2, placed inside the straw 3 and/or the nasal spray 11, which is able to be connected by means of Bluetooth to a latest-generation cellular device, CGM and insulin pump, receives information and allows the administration of the exact quantities to be administered as well as the preservation and containment of the glucose and/or glucagon contained within the aforesaid body of the device 1.

The present bracelet device is closed by means of the aforesaid magnetic closure 5 which is formed by two components: at least a metallic component placed on the right end 1.1 set on at least a magnetic component placed on the left end 1.2 and at least a magnetic component set on at least a metallic component placed on the right end 1.1.

The aforesaid bracelet device is also reloaded with glucose and/or glucagon by means of the aforesaid reload cartridge 6 comprising at least a tank 6.2 designed for containing predetermined quantities of glucose and/or glucagon and at least a lip 6.1 which allows the insertion of the aforesaid reload cartridge within the body of the device 1 through the aforesaid reload valve 4.

On the aforesaid body of the device 1, a display 7 is placed having preferably rectangular shape, which integrates a movement and/or touch interaction technology; such display 7 is designed for providing a plurality of data to the user, as it is also able to interact with the aforesaid smart valve 2, with latest-generation cellular device, CGM and insulin pumps. The display 7 can then communicate with a glycemia detector 8, also placed on said body of the device 1 and designed for checking the quantity of glucose in the blood and in turn communicating with the aforesaid display 7.

The present bracelet device is capable of communicating with multiple smartphones, allowing remote interactions and it integrates a geolocation device and SOS messaging in order to confront difficult moments as well as be able to produce sound alarms and vibrations.

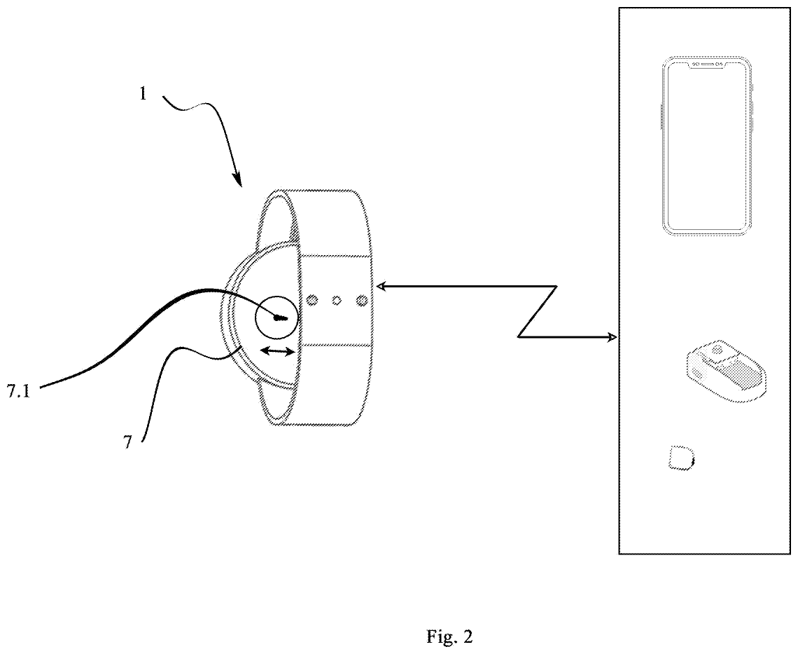

As shown in FIG. 2, near the aforesaid display 7 an injection device 7.1 is placed that is constituted by a retractable needle adapted to act as syringe actuatable by means of the display 7 itself or even remotely through the use of a suitable developed application. In addition, the bracelet device is designed for being personalized upon request of any user, in a sport or luxury line or made of any other shape or color, in addition to the fact of being made with recycled materials and/or of natural origin designed for being able to fall within a circular economy. Alternatively, the present bracelet device is impermeable, fireproof and underwater, as well as rechargeable by means of solar energy. Finally, the bracelet device is thermoregulated and has dark color so as to prevent the deterioration of the liquid at its interior and to maintain a suitable and constant temperature so to avoid crystallizations and oxidations of the glucose.

1. Ergonomic, smart wearable device, containing glucose and/or glucagon adapted to restore the concentration of glucose in the blood of a user; said wearable device being adapted to contain predetermined quantities of glucose to be administered in case of episodes of hypoglycemia to any one user; said wearable device comprising:
at least one body of the device with preferably tubular shape or in any case adapted to contain predetermined quantities of glucose; said body of the device being adapted to be worn on the wrist of the user and fixed by means of a closure; said closure being adapted to join the two ends of said body of the device; said body of the device comprising at least a reload valve which allows reinserting the predetermined quantity of glucose, preventing the accidental leakage thereof; —at least said reload valve adapted to house at least a lip of a reload cartridge; said valve being adapted to be traversed by at least a said reload cartridge and being adapted for containing and preserving glucose within said body of the device;
at least one straw, place above said body of the device, adapted for the user to consume a predetermined quantity of glucose; said at least one straw being reversibly liftable from said body of the device, allowing the oral administration of glucose for at least a user;
at least one smart valve placed inside said at least one straw being adapted to be connected by wireless communication to a latest-generation cellular device, so as to receive information regarding the exact quantity to be administered to any one user and adapted for preserving and containing the glucose contained within said body of the device;
at least one said magnetic closure, comprising two components, at least one metallic component placed on the right end adapted to lie on at least a magnetic component placed on the left end and at least one magnetic component adapted to lie on at least a metallic component placed on the right end; said closure being adapted to close the two said ends of said body of the device;
at least one said reload cartridge comprising at least a tank adapted to contain predetermined quantities of glucose and at least a lip adapted to enter, by means of said reload valve, within said device body, allowing the restoration of glucose;
at least one display placed on said body of the device, integrating movement and/or touch interaction technology; said display being adapted to provide a plurality of data and being adapted to interact with said smart valve;
at least one glycemia detector N-placed on said body of the device adapted to check the quantity of glucose in the blood of the user adapted to interact with said display.

2. Ergonomic, smart wearable device, containing glucose and/or glucagon adapted to restore the concentration of glucose in the blood of a user; said wearable device being adapted to contain predetermined quantities of glucagon to be administered in case of episodes of severe hypoglycemia to any one user; said wearable device comprising:
at least one body of the device with preferably tubular form or in any case adapted to contain predetermined quantities of glucagon; said body of the device being adapted to be worn on the wrist of the user and fixed by means of a closure; said closure being adapted to join the two ends of said body of the device; said body of the device comprising at least one a reload valve which allows restoring the predetermined quantity of glucagon, preventing the accidental leakage thereof;
at least said reload valve placed close to said closure adapted to house at least a lip of a reload cartridge; said valve being adapted to be traversed by at least a said reload cartridge and being adapted for containing and preserving glucagon within said body of the device;
at least one retractable nasal spray, placed above said body of the device, adapted for the user to consume a predetermined quantity of glucagon; said nasal spray being retractable by said body of the device, allowing the administration of glucagon through nose pathways to at least a user;
at least one smart valve placed inside said nasal spray, being adapted to be connected by wireless communication to a latest-generation electronic device, so as to receive information regarding the exact quantity to be administered to any one user and adapted for preserving the glucagon contained within said body of the device;
at least one said magnetic closure, comprising two components, at least a metallic component placed on the right end adapted to lie on at least a magnetic component placed on the left end and at least a magnetic component adapted to lie on at least a metallic component placed on the right end; said closure being adapted to close the two said ends of said body of the device;
at least one said reload cartridge comprising at least a tank adapted to contain predetermined quantities of glucagon and at least a lip adapted to enter by means of said reload valve within said device body, allowing the restoration of glucagon;
at least one display placed on said body of the device, integrating movement and/or touch interaction technology; said display being adapted to provide a plurality of data and being adapted to interact with said smart valve; and
at least one glycemia detector placed on said body of the device adapted to check the quantity of glucose in the blood of the user adapted to interact with said display.

3. Smart bracelet device containing glucose and glucagon, adapted to restore the concentration of glucose in the blood of a user; said bracelet device being adapted to contain predetermined quantities of glucose and glucagon to be administered in case of episodes of hypoglycemia or severe hypoglycemia to any one user; said bracelet device comprising two separate internal tanks: one filled with glucose and a second filled with glucagon; said bracelet device being provided with the wearable device of claim 1, comprising said nasal spray at the tank of said glucagon and at least one said straw at the tank of said glucose.

4. Smart bracelet device containing glucose and/or glucagon, according to claim 3, further comprising means for interconnection with one or more smartphones, CGM and insulin pumps and allowing remote interactions.

5. Smart bracelet device containing glucose and/or glucagon, according to claim 3 further comprising a geolocation device and SOS messaging system for signaling in difficult conditions, as well as sound alarms and vibrations for signals directed to the user.

6. Smart bracelet device containing glucose and/or glucagon, according to claim 3, further comprising at least an injection device, comprising a retractable needle, adapted to act as a syringe, manually controlled by means of said display or automatically controlled by any one latest-generation electronic device, adapted to inject predetermined quantities of glucose and/or glucagon as a function of the detected data regarding glucose in the blood of the user.

7. Smart bracelet device containing glucose and/or glucagon, according to claim 3, wherein the bracelet device is personalized upon request of each user and is made in a sport or luxury line.

8. Smart bracelet device containing glucose and/or glucagon, according to claim 3, wherein the bracelet device is made with recycled materials and natural materials that can be reused once worn, in order to fall within a circular economy.

9. Smart bracelet device containing glucose and/or glucagon, according to claim 3, further comprising solar charging means for recharging via solar energy.

10. Smart bracelet device containing glucose and/or glucagon, according to claim 3, further comprising data connection means so as to interact with a dedicated application installable on latest-generation electronic devices.

11. Smart bracelet device containing glucose and/or glucagon, according to claim 3, wherein said bracelet device is impermeable, underwater and fireproof so as to allow the use thereof in every condition, and has a dark color in order to prevent the deterioration of the glucose and/or glucagon due to solar rays.

12. Smart bracelet device containing glucose and/or glucagon, according to claim 3, further comprising thermoregulation means so as to maintain the temperature of the glucose and/or glucagon within pre-established parameters such to prevent the crystallization and oxidation of the glucose.

13. Smart bracelet device containing glucose and glucagon, adapted to restore the concentration of glucose in the blood of a user; said bracelet device being adapted to contain predetermined quantities of glucose and glucagon to be administered in case of episodes of hypoglycemia or severe hypoglycemia to any one user; said bracelet device comprising two separate internal tanks: one filled with glucose and a second filled with glucagon; said bracelet device being provided with the wearable device of claim 2, comprising said nasal spray at the tank of said glucagon and at least one said straw at the tank of said glucose.
